(19) United States
(12) Patent Application Publication
(10) Pub. No.: US 2022/0387279 A1
(43) Pub. Date: Dec. 8, 2022

(54) Oral Care Compositions

(71) Applicant: Colgate-Palmolive Company, New York, NY (US)

(72) Inventors: Murilo Nogueira NAKAJIMA, Sao Paulo (BR); Saide TANG, Princeton, NJ (US); Betty WON, Princeton Junction, NJ (US); Enzo UTIMA, Sao Paulo (BR); Guofeng XU, Plainsboro, NJ (US); Paloma PIMENTA, Staten Island, NY (US); Sarita Vera MELLO, Ringwood, NJ (US); Melissa MUIR, Lopatcong, NJ (US)

(73) Assignee: Colgate-Palmolive Company, New York, NY (US)

(21) Appl. No.: 17/825,365

(22) Filed: May 26, 2022

Related U.S. Application Data
(60) Provisional application No. 63/193,479, filed on May 26, 2021.

(51) Int. Cl. A61K 8/43; A61Q 11/00; A61K 8/73; A61K 8/49; A61K 8/04

(52) U.S. Cl. CPC A61K 8/43; A61Q 11/00; A61K 8/731; A61K 8/4926; A61K 8/042; A61K 2800/92; A61K 2800/872; A61K 2800/591

(57) ABSTRACT

This application provides, among other things, oral care compositions where the addition of a stabilizing amount of a nonionic gelling and thickening agent, (e.g., hydroxyethyl cellulose (HEC)), to formulations comprising a bisbiguanide (e.g., chlorhexidine (CHX)) agent allows for proper structure and consistency to the formulation and to allow it to be used as a gel or a toothpaste to facility delivery of chlorhexidine, among other actives, to the teeth or gums.

### CROSS-REFERENCE TO RELATED APPLICATIONS

This application claims the benefit of priority to U.S. Provisional Patent Application Ser. No. 63/193,479, filed May 26, 2021, the contents of which are incorporated herein by reference in its entirety.

### BACKGROUND

Biofilms form when bacteria adhere to surfaces in some form of watery environment and begin to excrete a slimy, glue-like substance that can stick to all kinds of materials—metals, plastics, soil particles, medical implant materials, biological tissues. Dental plaque is a biofilm that adheres to tooth and other oral surfaces, particularly at the gingival margin, and is implicated in the occurrence of gingivitis, periodontitis, caries and other forms of periodontal disease. Dental plaque is cohesive and highly resistant to removal from teeth and/or oral surfaces. Bacteria associated with dental plaque convert sugar to glucans, which are insoluble polysaccharides that provide plaque with its cohesive properties. Anaerobic bacteria in plaque metabolize sugar to produce acids which dissolve tooth minerals, damaging the enamel and eventually forming dental caries. Saliva can buffer acids produced by bacteria and promote remineralization of the enamel, but extensive plaque can block the saliva from contact with the enamel. Redeposition of minerals in the biofilm forms a hard deposit on the tooth called calculus (or tartar), which becomes a local irritant for the gums, causing gingivitis.

Various antibacterial agents can retard the growth of bacteria and thus reduce the formation of biofilm on oral surfaces. In many cases, these antibacterial agents are cationic, for example quaternary ammonium surfactants such as cetyl pyridinium chloride (CPC), bisbiguanides such as chlorhexidine, metal cations such as zinc or stannous ions, and guanidines such as arginine.

However, while those in the field have explored the use of certain antiseptic and bactericidal agents such as bisbiguanide agents (e.g., chlorhexidine) in oral care products (e.g., mouthwash), one challenge in these formulations is to stabilize it in a higher-complex system such as gels and toothpaste. Chlorhexidine can be a very sensitive compound which is often required to be in a positive-charged form to provide the therapeutic benefits. Anything in the formula, for example anionic compounds, some impurities coming from raw materials or certain conditions (pH), have the potential to deactivate chlorhexidine which can make it ineffective from a therapeutic standpoint.

Accordingly, there is a need for an oral care product that contains a bisbiguanide agent (e.g., chlorhexidine) that can be formulated in a higher-complex system such as gels and toothpaste and allow for effective delivery to a consumer.

### BRIEF SUMMARY

It is surprisingly found that the addition of a stabilizing amount of a nonionic gelling and thickening agent, e.g., a nonionic cellulose ether, (e.g., hydroxyethyl cellulose (HEC)), to formulations comprising a bisbiguanide (e.g., chlorhexidine (CHX)) agent allows for proper structure and consistency to the formulation to allow it to be used as a gel or a toothpaste and facilities delivery of a bisbiguanide (e.g., chlorhexidine) to the teeth or gums. For example, in one aspect, formulas containing 1.75% of HEC demonstrate acceptable consistency and structure while providing excellent CHX recovery (min. 90%) when it is added at 0.12% and 0.20%. In another aspect, formulas of the present invention containing 0.12% CHX and 0.20% CHX were submitted to aging tests and completed the studies (13 weeks) with satisfactory results. CHX levels remained within the specification (min. 90% recovery) and p-chloroaniline (pCA), a toxic compound, below 3 ppm.

The disclosure thus provides, in one embodiment, oral care compositions comprising:
((i) an effective amount of a bisbiguanide (e.g., chlorhexidine) (e.g., chlorhexidine digluconate) in free or orally acceptable salt form;
(ii) an effective amount of a nonionic cellulose ether (e.g., hydroxyethyl cellulose), (e.g., wherein the amount of nonionic cellulose ether is effective to stabilize the bisbiguanide); and
(iii) water.

The disclosure further provides methods of treating and/or inhibiting dental plaque, gingivitis, dental erosion, staining, and/or biofilm formation comprising administering to the oral cavity a composition as described in any of Composition 1, et seq. In one aspect, the oral care composition described herein would be used in a professional setting to help the patient who, for instance, underwent a surgery (as a post-oral surgery) or is in need of intense care due to severe gum conditions and thus requiring product application on targeted areas.

In one aspect, the compositions of the would be for professional administration to a patient who, for instance, underwent a surgery (as a post-oral surgery) or is in need of intense care due to severe gum conditions and thus requiring product application on targeted area. In some aspects, the compositions of the present invention (e.g., any of Composition 1 et seq) is in the form of a gel that can be delivered using a syringe and/or gel pen applicator.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight of the total composition. Unless specified otherwise, the amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In a first embodiment, the disclosure provides oral care compositions (Composition 1) comprising:
(i) an effective amount of a bisbiguanide (e.g., a cationic bisbiguanide) (e.g., chlorhexidine digluconate) in free or orally acceptable salt form;
(ii) an effective amount of a nonionic cellulose ether (e.g., hydroxyethyl cellulose), (e.g., wherein the amount of nonionic cellulose ether is effective to stabilize the bisbiguanide); and
(iii) water.

For example, the disclosure provides embodiments of Composition 1 as follows:
1.1 Composition 1, wherein the bisbiguanide (e.g., cationic bisbiguanide) is selected from chlorhexidine (e.g., chlorhexidine digluconate), poly(hexamethylene) biguanide (e.g., polyhexanide).
1.2 Composition 1.1, wherein the bisbiguanide is chlorhexidine in free or orally acceptable salt form (e.g., chlorhexidine gluconate) (e.g., from 0.05%-3% by wt.) (e.g., from 0.1%-2% by wt.) (e.g., about 0.12% by wt. of the total composition) (e.g., about 0.2% of the total composition)
1.3 Composition 1-1.2 wherein the bisbiguanide is a cationic bisbiguanide in orally acceptable salt form.
1.4 Any of the preceding compositions wherein the bisbiguanide is chlorhexidine digluconate (e.g., from 0.1%-0.3% by wt.) (e.g., about 0.12% by wt.) (e.g., about 0.20% by wt.).
1.5 Any of the preceding compositions wherein the composition comprises an orally acceptable cationic active agent selected from one or more of: quaternary ammonium surfactants (e.g., a pyridinium surfactant) (e.g., cetyl pyridinium chloride (CPC)), amino acids (e.g, arginine), metal cations (e.g., zinc, calcium, or stannous ions), guanidinium polymers, and combinations thereof.
1.6 The Composition of 1.5, wherein the orally acceptable cationic active agent comprises an agent selected from one or more of: cetyl pyridinium chloride (CPC)); arginine (e.g, in free or salt form); antimicrobial guanidinium polymers; a source of zinc (e.g., zinc citrate, zinc oxide, zinc lactate, zinc phosphate, or combinations thereof), and combinations thereof.
1.7 Any foregoing composition wherein the orally acceptable cationic active agent comprises a pyridinium surfactant, e.g., cetyl pyridinium chloride (CPC).
1.8 Any foregoing composition wherein the orally acceptable cationic active agent comprises cetyl pyridinium chloride (CPC).
1.9 Any foregoing composition wherein the orally acceptable cationic active agent comprises arginine.
1.10 Any foregoing composition wherein the orally acceptable cationic active agent comprises a source of zinc ions.
1.11 The composition of 1.10 wherein the source of zinc ions is selected from the group consisting of zinc citrate, zinc lactate, zinc phosphate, and zinc oxide (e.g., wherein the zinc ion source comprises zinc citrate).
1.12 Any foregoing composition wherein the orally acceptable cationic active agent comprises a source of stannous ions.
1.13 The composition of 1.12 wherein the source of zinc ions is selected from the group consisting of stannous fluoride, stannous pyrophosphate and stannous chloride.
1.14 Any foregoing composition wherein the comprises cetyl pyridinium chloride, in an amount of 0.01 to 0.1%, e.g., about 0.015% by wt. of the total composition.
1.15 Any foregoing composition wherein the effective amount of the bisbiguanide, in free or salt form, is present and comprises chlorhexidine digluconate, in an amount of 0.1 to 0.3% by wt. of the total composition, e.g., about 0.12%, e.g., about 0.2%.
1.16 Any of the foregoing compositions, wherein the nonionic cellulose ether is selected from the group consisting of: ethyl cellulose, hydroxyethyl cellulose, methyl cellulose, sodium carboxy methyl cellulose, and benzyl cellulose.
1.17 Any of the foregoing compositions, wherein the nonionic cellulose ether comprises hydroxyethyl cellulose (HEC).
1.18 The preceding composition, wherein the hydroxyethyl cellulose is in an amount of from 0.5 wt. % to 3 wt. % of the total composition.
1.19 The preceding composition, wherein the hydroxyethyl cellulose is in an amount of from 0.5 wt. % to 2 wt. % of the total composition.
1.20 The preceding composition, wherein the hydroxyethyl cellulose is in an amount of from 1 wt. % to 2 wt. % of the total composition.
1.21 The preceding composition, wherein the hydroxyethyl cellulose is in an amount of from 1.5 wt. % to 2 wt. % of the total composition.
1.22 The preceding composition, wherein the hydroxyethyl cellulose is in an amount of from about 1.75 wt. % of the total composition.
1.23 Any of the preceding composition, wherein the composition further comprises a humectant.
1.24 The preceding composition, wherein the humectant comprises glycerin, or sorbitol, or propylene glycol, or combinations thereof.
1.25 The preceding composition, wherein the composition comprises glycerin.
1.26 Any of the preceding compositions, wherein the glycerin is in an amount from 1%-20% by wt. of the composition.
1.27 The composition of 1.26, wherein the glycerin is in an amount from 3%-10% by wt. of the composition.
1.28 The composition of 1.27, wherein the glycerin is about 5% by wt. of the composition.
1.29 The composition of 1.27, wherein the glycerin is about 7% by wt. of the composition.
1.30 Any of the preceding compositions, wherein the composition further comprises sorbitol.
1.31 The preceding composition, wherein the sorbitol is from 5%-15% by wt. of the composition.
1.32 The preceding composition, wherein the sorbitol is from 5%-10% by wt. of the composition.
1.33 The preceding composition, wherein the sorbitol is from 6.5%-7% by wt. of the composition.
1.34 The composition of 1.32, wherein the sorbitol is from 8%-9% by wt. of the total composition.
1.35 Any of the preceding compositions comprising sorbitol and glycerin.
1.36 The preceding composition wherein the composition comprises from 5%-10% by wt. of sorbitol and from 3%-10% by wt. of glycerin, wherein the wt. % is relative to the total weight of the composition.
1.37 Any of the preceding compositions wherein the humectant comprises propylene glycol (e.g. from 5%-10% by wt. of the total composition).
1.38 Any foregoing composition wherein the anionic surfactant comprises an alkyl sulfate or an alkyl ether sulfate in free or orally acceptable salt form.
1.39 Any foregoing composition wherein the anionic surfactant comprises a sodium, potassium, ammonium, and ethanolammonium salts of linear C8-C18 alkyl sulfate or C8-C18 alkyl ether sulfate.
1.40 Any foregoing composition wherein the anionic surfactant comprises sodium, ether sulfate (SLES), sodium lauryl sulfate, and ammonium lauryl sulfate.
1.41 Any foregoing composition wherein the anionic surfactant comprises sodium lauryl sulfate.
1.42 Any foregoing composition wherein the anionic surfactant is present in an amount of 0.01 to 5.0%, 0.1 to 2.0%, 0.1 to 1.0%, 0.2 to 0.4%, or about 0.33%.
1.43 Any foregoing composition further comprising a anionic surfactant.
1.44 Any foregoing composition comprising a nonionic surfactant selected from poloxamers or polyoxyethylene, e.g., poloxarner 407.
1.45 Any foregoing composition comprising a nonionic surfactant which is a block copolymer of polyethylene glycol and polypropylene glycol.
1.46 Any foregoing composition comprising a nonionic surfactant in an amount of about 0.01 to 5.0% by wt. of the total composition.
1.47 Any foregoing composition further comprising an amino acid or a polyamine, in free or orally acceptable salt form.
1.48 Any foregoing composition wherein the composition comprises 50% to 95% water by wt. of the composition.
1.49 Any foregoing composition wherein the composition comprises 70%-90% water by wt. of the composition.
1.50 Any foregoing composition wherein the compositions comprises 65%-80% water by wt. of the composition.
1.51 Any foregoing composition wherein the composition comprises one or more of a thickener, a buffer, a humectant, a surfactant, an abrasive, a sweetener, a flavorant, a pigment, a dye, an anti-caries agent, an anti-bacterial agent, a whitening agent, a desensitizing agent, a preservative, or a mixture thereof.
1.52 Any foregoing composition wherein the composition comprises a phosphate buffer.
1.53 Any foregoing composition wherein the composition comprises a buffer wherein the buffer comprises sodium hydroxide.
1.54 Any foregoing composition further comprising a pH adjustment agent selected from lactic acid, citric acid, hydrochloric acid, glycolic acid, sodium hydroxide, potassium chloride, monosodium citrate, disodium citrate, monosodium malate, sodium carbonate, bicarbonates, sesquicarbonate, borates, silicates, monosodium phosphate, trisodium phosphate, pyrophosphate salts, imidazole, or combinations thereof; e.g., citric acid.
1.55 Any foregoing composition comprising a pH adjustment agent in an amount of 0.0001% to 1.0%.
1.56 The preceding composition, wherein the pH adjustment agent is citric acid.
1.57 Any foregoing composition wherein the composition has a pH of about 1 to 7, about 3 to 6, about 5 to 6, or about 5.25 to 5.75.
1.58 Any foregoing composition wherein the composition comprises an abrasive.
1.59 Any foregoing composition wherein the composition comprises an abrasive, wherein the abrasive comprises silica.
1.60 Any of the forgoing compositions which comprises a non-silica abrasive.
1.61 Any foregoing composition wherein the composition a sweetener.
1.62 Any foregoing composition wherein the composition a sweetener, wherein the sweetener is sodium saccharin.
1.63 Any foregoing composition wherein the composition comprises a flavorant.
1.64 Any foregoing composition wherein the composition comprises a dye, e.g., FD&C Blue No. 1.
1.65 Any foregoing composition wherein the composition comprises an anti-caries agent.
1.66 Any foregoing composition wherein the composition comprises a fluoride ion source.
1.67 Any foregoing composition wherein the composition comprises a fluoride ion source, wherein the fluoride ion source is stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride) ammonium fluoride, titanium fluoride, hexafluorosulfate, or a mixture thereof.
1.68 Any foregoing composition wherein the composition comprises a whitening agent.
1.69 Any foregoing composition wherein the composition comprises a whitening agent, wherein the whitening agent is hydrogen peroxide.
1.70 Any foregoing composition wherein the composition comprises a desensitizing agent, a vitamin, a preservative, an enzyme, or a mixture thereof.
1.71 Any foregoing composition wherein the composition is selected from: a mouthwash, toothpaste, tooth gel, tooth powder, non-abrasive gel, mousse, foam, mouth spray, lozenge, oral tablet, and dental implement.
1.72 Any foregoing composition wherein the composition is a tooth gel.
1.73 Any of the foregoing compositions wherein the oral care composition is a gel (e.g., tooth gel) and comprises:
   An effective amount of chlorhexidine in free or orally acceptable salt forms (e.g., chlorhexidine gluconate or chlorhexidine digluconate);
   Cetylpyridinium chloride;
   An effective amount of hydroxyethyl cellulose (e.g., wherein the amount of hydroxethyl cellulose ether is effective to stabilize the bisbiguanide); and

wherein the water content is from 60%-90% by wt. of the composition.
1.74 Any of the foregoing compositions wherein the oral care composition is a gel (e.g., tooth gel) and comprises:
   An effective amount of chlorhexidine in free or orally acceptable salt form (e.g., chlorhexidine gluconate or chlorhexidine digluconate);
   Cetylpyridinium chloride;
   An effective amount of hydroxyethyl cellulose (e.g., wherein the amount of hydroxethyl cellulose ether is effective to stabilize the bisbiguanide);
   Glycerin and Sorbitol; and

wherein the water content is from 60%-90% by wt. of the composition.
1.75 Any of the foregoing compositions wherein the oral care composition is a gel (e.g., tooth gel) and comprises:
   Chlorhexidine in free or orally acceptable salt form (e.g., chlorhexidine gluconate or chlorhexidine digluconate) in an amount from 0.1%-2% by wt. of the total composition (e.g., from 0.05%-3% by wt.) (e.g., from 0.1%-2% by wt.) (e.g., about 0.12% by wt.) (e.g., about 0.2% by wt.);
   Cetylpyridinium chloride from 0.01 to 0.1% by wt. of the total composition (e.g., about 0.015%);
   An effective amount of hydroxyethyl cellulose (e.g., wherein the amount of hydroxethyl cellulose ether is effective to stabilize the bisbiguanide), wherein the hydroxyethyl cellulose is in an amount of from 1.5 wt. % to 2 wt. % of the total composition (e.g., about 1.75% by wt.);
   Glycerin and Sorbitol; and

wherein the water content is from 60%-90% by wt. of the composition.
1.76 Any of the foregoing compositions wherein the oral care composition is a gel (e.g., tooth gel) and comprises:
   Chlorhexidine in free or orally acceptable salt form (e.g., chlorhexidine gluconate or chlorhexidine digluconate) in an amount of about 0.12% by wt. of the total composition;
   Cetylpyridinium chloride from 0.01 to 0.1% by wt. of the total composition (e.g., about 0.015%);
   An effective amount of hydroxyethyl cellulose (e.g., wherein the amount of hydroxethyl cellulose ether is effective to stabilize the bisbiguanide), wherein the hydroxyethyl cellulose is in an amount of from 1.5 wt. % to 2 wt. % of the total composition (e.g., about 1.75% by wt.);
   Glycerin and Sorbitol; and

wherein the water content is from 60%-90% by wt. of the composition
1.77 Any of the foregoing compositions wherein the oral care composition is a gel (e.g., tooth gel) and comprises:
   Chlorhexidine in free or orally acceptable salt form (e.g., chlorhexidine gluconate or chlorhexidine digluconate) in an amount of about 0.20% by wt. of the total composition;
   Cetylpyridinium chloride from 0.01 to 0.1% by wt. of the total composition (e.g., about 0.015%);
   An effective amount of hydroxyethyl cellulose (e.g., wherein the amount of hydroxethyl cellulose ether is effective to stabilize the bisbiguanide), wherein the hydroxyethyl cellulose is in an amount of from 1.5 wt. % to 2 wt. % of the total composition (e.g., about 1.75% by wt.);
   Glycerin and Sorbitol; and

wherein the water content is from 60%-90% by wt. of the composition
1.78 Any preceding composition, wherein the composition is formulated to be free of silicas and/or silica abrasives.
1.79 Any of the foregoing compositions wherein the oral care composition is a viscous liquid (e.g., gel)(e.g., dental or tooth gel).
1.80 Any of the foregoing compositions wherein the oral care composition is a viscous liquid (e.g., gel) (e.g., dental or tooth gel) that maintains its consistency during storage (e.g., enabling the product to be applied to the tooth surface).
1.81 Any of the foregoing compositions wherein the oral care composition is a gel packaged, and delivered to a patient in need thereof, in a soft applicator dental pen, syringe or brush.
1.82 Any of the foregoing compositions wherein the oral care composition is a gel that is delivered via a syringe and/or dental pen delivery system.
1.83 Any of the foregoing compositions wherein the oral care composition is in the form of a viscoelastic fluid.
1.84 Any of the foregoing compositions wherein the bisbiguanide is chlorhexidine, and wherein the chlorhexidine is the only bisbiguanide source in the composition.
1.85 Any of the foregoing compositions wherein the nonionic cellulose ether is hydroxyethyl cellulose, and wherein the HEC is the only nonionic cellulose ether in the composition.
1.86 Any foregoing composition for use in any of Methods A-E.
1.87 Any of the foregoing composition for use in a patient that has undergone surgery (e.g., post-surgery).
1.88 Any of the foregoing compositions, wherein chlorhexidine is present as chlorhexidine digluconate in an amount from 0.05%-0.3% by wt. of the total composition.
1.89 Any of the foregoing compositions, wherein chlorhexidine is present as chlorhexidine digluconate in an amount of about 0.12% by wt. of the total composition.
1.90 Any of the foregoing compositions, wherein chlorhexidine is present as chlorhexidine digluconate in an amount of about 0.20% by wt. of the total composition.
1.91 Any of the foregoing compositions wherein the composition is in the form of a gel (e.g., a spot gel) that can be applied to a targeted or specific area.
1.92 Any of the foregoing compositions, wherein the composition comprises a source of zinc ions (e.g., zinc citrate) from 0.1%-2.5% by wt. of the composition (e.g., zinc citrate at 0.5% by wt. of the composition).
1.93 Any of the foregoing compositions wherein the oral care composition is a gel (e.g., tooth gel) wherein the composition comprises from 0.1%-0.3% by wt. of chlorhexidine gluconate relative to the total weight of the composition (e.g., about 0.12% by wt. of chlorhexidine gluconate) (e.g., about 0.2% by wt. of chlorhexidine gluconate).
1.94 Any of the foregoing compositions wherein the oral care composition is a gel (e.g., tooth gel) wherein the composition comprises from 0.1%-0.3% by wt. of chlorhexidine gluconate relative to the total weight of the composition (e.g., about 0.12% by wt. of chlorhexidine gluconate) (e.g., about 0.2% by wt. of chlorhexidine gluconate).
1.95 Any of the foregoing compositions wherein the oral care composition is a gel (e.g., tooth gel) and comprises:
   An effective amount of chlorhexidine in free or orally acceptable salt form (e.g., chlorhexidine gluconate or chlorhexidine digluconate from 0.05%-0.25% by wt., or about 0.12% by wt., or about 0.20% by wt.);
   An effective amount of hydroxyethyl cellulose (e.g., wherein the amount of hydroxethyl cellulose ether is effective to stabilize the bisbiguanide) (from 0.5 wt. % to 3 wt. % of the total composition); and

wherein the water content is from 50%-90% by wt. of the composition.
1.96 Any of the foregoing oral care compositions, wherein the oral care composition comprises chlorhexidine in free or orally acceptable salt form (e.g., from 0.05%-3% by wt. of the total composition) (e.g., from 0.04%— 0.3% by wt. of the total composition) (e.g., from 0.1%-2% by wt. of the total composition) (e.g., about 0.12% by wt.) (e.g., about 0.2% by wt. of the total composition).
1.97 The oral care composition of 1.96, wherein the chlorhexidine is a salt selected from: chlorhexidine gluconate (or chlorhexidine digluconate), chlorhexidine acetate, chlorhexidine diacetate, chlorhexidine hydrochloride, and chlorhexidine dihydrochloride.
1.98 The oral care composition of any of the preceding oral care compositions, wherein the composition comprises chlorhexidine in orally acceptable salt form (e.g., chlorhexidine gluconate or chlorhexidine digluconate) (e.g., from 0.05%-0.3% by wt.) (e.g., about 0.12% by wt.) (e.g., about 0.2% by wt.) and wherein the amount of the chlorhexidine is measured as the amount of the salt relative to the weight of the total composition.

Any of Composition 1 et seq where the composition is administered to a patient in need thereof who, e.g., has undergone a surgery (e.g., post-oral surgery).

Any of Composition 1 et seq where the composition is administered to a patient in need thereof wherein the patient in need thereof has severe gum conditions and wherein the product is applied to a targeted or specific area (e.g., using a pen system to deliver a gel).

In yet a further aspect, the invention contemplates a Delivery System (Delivery System 1) for administration of any of Composition 1, et seq to a patient in need thereof. In one aspect the delivery system comprises any of Composition 1, et seq, wherein the composition is a gel. In another aspect, the delivery system comprises a syringe for administration of the composition of any of Composition 1, et seq (e.g., wherein the syringe is used by a professional).

In another aspect the delivery system comprises both a syringe and a dental pen for administration of any of Composition 1, et seq.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

As used herein, “viscoelastic fluid” refers to a complex fluid that exhibits mechanical properties that are both elastic (solid-like, e.g., rubber) and viscous (liquid-like or flowable, e.g., water). A viscoelastic fluid composition may deform and flow under the influence of an applied shear stress (e.g., shaking or swishing in the mouth), but when the stress is removed, the composition will recover the deformation.

As used herein, “CHX” refers to chlorhexidine. As used herein “chlorhexidine gluconate” and “chlorhexidine digluconate” are used interchangeably, wherein the formula of chlorhexidine gluconate or chlorhexidine digluconate refers to: (1,1′-hexamethylene bis [5-(p-chlorophenyl) biguanide]di-D-gluconate).

In some embodiments the compositions of the disclosure, e.g., any of Composition 1 et seq, comprises an orally acceptable cationic active agent. As used herein, “orally acceptable cationic active agent” means an agent which is cationic in aqueous solution at neutral pH and which provides some benefit, e.g. antimicrobial, antigingivitis, and/or antierosion activity, to the teeth or oral cavity. While in aqueous formulation, the agent will generally be in solution, but it may be introduced to the formulation formulated in free or orally acceptable salt form. In certain embodiments, the orally acceptable cationic active agent is selected from one or more of quaternary ammonium surfactants (such as cetyl pyridinium chloride (CPC)), cationic amino acids (such as arginine), metal cations (such as zinc, calcium, or stannous ions), or combinations thereof.

In some embodiments the compositions of the disclosure, e.g., any of Composition 1 et seq, comprises an anionic surfactant. As used herein, “anionic surfactant” means those surface-active or detergent compounds that contain an organic hydrophobic group containing generally 8 to 26 carbon atoms or generally 10 to 18 carbon atoms in their molecular structure and at least one water-solubilizing group selected from sulfonate, sulfate, and carboxylate so as to form a water-soluble detergent. Usually, the hydrophobic group will comprise a C₈-C₂₂ alkyl, or acyl group. Such surfactants are employed in the form of water-soluble salts and the salt-forming cation usually is selected from sodium, potassium, ammonium, magnesium and mono-, di- or tri-C₂-C₃ alkanolammonium, with the sodium, magnesium and ammonium cations again being the usual ones chosen. Some examples of suitable anionic surfactants include, but are not limited to, the sodium, potassium, ammonium, and ethanolammonium salts of linear C₈-C₁₈ alkyl ether sulfates, ether sulfates, and salts thereof. Suitable anionic ether sulfates have the formula R(OC₂H₄)ₙ OSO₃M wherein n is 1 to 12, or 1 to 5, and R is an alkyl, alkylaryl, acyl, or alkenyl group having 8 to 18 carbon atoms, for example, an alkyl group of C₁₂-C₁₄ or C₁₂-C₁₆, and M is a solubilizing cation selected from sodium, potassium, ammonium, magnesium and mono-, di- and triethanol ammonium ions. Exemplary alkyl ether sulfates contain 12 to 15 carbon atoms in the alkyl groups thereof, e.g., sodium laureth (2 EO) sulfate. Some preferred exemplary anionic surfactants that may be used in the compositions of the present disclosure include sodium laurel ether sulfate (SLES), sodium lauryl sulfate, and ammonium lauryl sulfate. In certain embodiments, the anionic surfactant is present in an amount of 0.01 to 5.0%, 0.1 to 2.0%, 0.2 to 0.4%, or about 0.33%.

In some embodiments the compositions of the disclosure, e.g., any of Composition 1 et seq, comprises a nonionic surfactant. As used herein, “nonionic surfactant” generally refers to compounds produced by the condensation of alkylene oxide groups (hydrophilic in nature) with an organic hydrophobic compound which may be aliphatic or alkyl-aromatic in nature. Examples of suitable nonionic surfactants include polyethylene glycol (e.g., PEG-40 hydrogenated castor oil), poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, alkyl polyglycosides (for example, fatty alcohol ethers of polyglycosides, such as fatty alcohol ethers of polyglucosides, e.g., decyl, lauryl, capryl, caprylyl, myristyl, stearyl and other ethers of glucose and polyglucoside polymers, including mixed ethers such as capryl/caprylyl (C₈₋₁₀) glucoside, coco (C₈₋₁₆) glucoside, and lauryl (C₁₂₋₁₆) glucoside), long chain tertiary amine oxides, long chain tertiary phosphine oxides, long chain dialkyl sulfoxides, and mixtures of such materials.

In some embodiments the compositions of the disclosure, e.g., any of Composition 1 et seq, comprises a nonionic surfactant selected from the group consisting of: amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and mixtures thereof. Examples of fatty acid amides include, but are not limited to, cocomonoethanolamide, lauramide monoethanolamide, cocodiethanolamide, and mixtures thereof. In certain embodiments, the nonionic surfactant is a combination of an amine oxide and a fatty acid amide. In certain embodiments, the amine oxide is a mixture of laurylamidopropyl dimethylamine oxide and myristylamidopropyl dimethylamine oxide. In certain embodiments, the nonionic surfactant is a combination of lauryl/myristylamidopropyl dimethylamine oxide and cocomonoethanolamide. In certain embodiments, the nonionic surfactant is present in an amount of 0.01 to 5.0%, 0.1 to 2.0%, 0.1 to 0.6%, 0.2 to 0.4%, about 0.2%, or about 0.5%

In some embodiments the compositions of the disclosure, e.g., any of Composition 1 et seq, can comprise a basic or neutral amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

In another aspect, the compositions of the invention (e.g., any of Compositions 1 et seq) can include a neutral amino acid, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

In some embodiments the compositions of the disclosure, e.g., any of Composition 1 et seq, comprises a tartar control agent. As used herein, a “tartar control agent” refers to a compound or a mixture of compounds that inhibit the formation of tartar, a mixture of calcium phosphates on organic matrices, and/or the deposition of plaque on teeth to form tartar (calculus).

In some embodiments the compositions of the disclosure, e.g., any of Composition 1 et seq, is administered as part of a method to treat or reduce staining of the enamel. As used herein, “staining” refers to a discoloration of a dental surface caused by adsorption or absorption of a color agent on or into the surface, or caused by reaction of material of the dental surface (e.g., dental enamel) with a color or noncolor agent contacting the surface.

As used herein, “dental surface” refers to a surface of a natural tooth or a hard surface of artificial dentition including a crown, cap, filling, bridge, dental implant and the like. In some embodiments, the dental surface is a natural tooth.

In some embodiments the compositions of the disclosure, e.g., any of Composition 1 et seq, are tooth gels. Any of the compositions of Composition 1, et seq. is suitable for oral care use, provided the ingredients are orally acceptable. In some embodiments, any of the compositions of the disclosure are a tooth gel, e.g, any of Composition 1, wherein the tooth gel comprises an effective amount of an orally acceptable bisbiguanide (e.g., chlorhexidine) (e.g., chlorhexidine digluconate), which is an antimicrobial, antigingivitis, anti-erosion and/or anti-caries agent, a nonionic cellulose ether (e.g., hydroxyethyl cellulose), and a pyridinium surfactant (e.g., cetyl pyridinium chloride), wherein the nonionic cellulose ether is in an amount effective to stabilize the bisbiguanide in the tooth gel.

The oral care composition used in the present disclosure can comprise significant levels of water. Water employed in the preparation of commercial oral compositions should be deionized and free of organic impurities. The amount of water in the compositions includes the free water that is added plus that amount which is introduced with other materials.

In certain embodiments the compositions of the disclosure, e.g., any of Composition 1 et seq, can comprise a humectant. Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case. In some embodiments, humectants are present at levels of 5% to 30%, e.g., 10% to 20% by weight.

In certain embodiments the compositions of the disclosure, e.g., any of Composition 1 et seq, can comprise a flavoring. Flavorings for use in the present invention may include extracts or oils from flavorful plants such as peppermint, spearmint, cinnamon, wintergreen, and combinations thereof, cooling agents such as menthol, methyl salicylate, as well as sweeteners, which may include polyols (which also function as humectants), saccharin, acesulfame, aspartame, neotame, stevia and sucralose.

Further provided is a method (Method A) for the treatment and/or inhibition of a gingivitis, plaque, and/or tartar on a dental surface, comprising contacting the dental surface with any of the preceding oral care compositions.

Further provided herein is Method A as follows:
A.1 Method A wherein the composition is Composition 1, e.g., selected from any of Compositions 1.1-1.97.
A.2 Method A or A.1 wherein the method is for the treatment of gingivitis, plaque, and/or tartar on the dental surface.
A.3 Method A.2, wherein the method is for the treatment of gingivitis.
A.4 Method A.2 wherein the method is for the treatment of staining on the dental surface.
A.5 Method A.2 wherein the method is for the treatment of plaque on the dental surface.
A.6 Method A.2 wherein the method is for the treatment of tartar on the dental surface.
A.7 Method A or A.1 wherein the method is for the inhibition of plaque, and/or tartar on the dental surface.
A.8 Method A.6 wherein the method is for the inhibition of a chemical stain on the dental surface.
A.9 Method A.6 wherein the method is for the inhibition of plaque on the dental surface.
A.10 Method A.6 wherein the method is for the inhibition of tartar on the dental surface.
A.11 Method A or A.1-A.9 wherein the dental surface is a human tooth.
A.12 Method A or A.1-A.10 wherein the composition is contacted with the dental surface by brushing.
A.13 Method A or A.1-A.10 wherein the composition is contacted with the dental surface by a dental pen or syringe.

Further provided is a method (Method B) for the treatment and/or inhibition of gum disease comprising contacting the oral cavity with any of the preceding oral care compositions.

Further provided herein is Method B as follows:
B.1 Method B wherein the composition is Composition 1, e.g., any of Compositions 1.1-1.97.
B.2 Method B or B.1 wherein the method is for the treatment of gum disease.
B.3 Method B, B.1, or B.2 wherein the gum disease is gingivitis.
B.4 Method B, B.1, or B wherein the gum disease is periodontitis.
B.5 Method B or B.1 wherein the method is for the inhibition of gum disease.
B.6 Method B, B.1, or B.5 wherein the gum disease is gingivitis.
B.7 Method B, B.1, or B.5 wherein the gum disease is periodontitis.
B.8 Method B or B.1-B.7 wherein the oral cavity is a human oral cavity.
B.9 Method B or B.1-B.8 wherein the composition is contacted with the oral cavity by brushing.
B.10 Method B or B.1-B.8 wherein the composition is contacted with the dental surface by a dental pen or syringe.

Further provided is a method (Method C) for the treatment and/or inhibition of halitosis comprising contacting the oral cavity with any of the preceding oral care compositions.

Further provided herein is Method C as follows:
C.1 Method C wherein the composition is Composition 1, e.g., any of Compositions 1.1-1.97.
C.2 Method C or C.1 wherein the oral cavity is a human oral cavity.
C.3 Method C, C.1, or C.2 wherein the composition is contacted with the oral cavity by brushing.
C.4 Method C or C.1-C.2 wherein the composition is contacted with the dental surface by a dental pen or syringe.

Further provided is a method (Method D) for inhibiting biofilm formation on a dental surface comprising contacting the dental surface with any of the preceding oral care compositions.

Further provided herein is Method D as follows:
D.1 Method D wherein the composition is Composition 1, e.g., any of Compositions 1.1-1.97.
D.2 Method D or D.1 wherein the dental surface is a human tooth.
D.3 Method D, D.1, or D.2 wherein the composition is contacted with the dental surface by brushing.
D.4 Method D or D.1-D.2 wherein the composition is contacted with the dental surface by a dental pen or syringe.

Further provided is a method (Method E) for treating and/or inhibiting bacteria from aggregating and forming bigger colonies in an oral cavity comprising contacting the oral cavity with any of the preceding oral care compositions.

Further provided herein is Method E as follows:
E.1 Method E wherein the composition is Composition 1, e.g., any of Compositions 1.1-1.97.
E.2 Method E or E.1 wherein the oral cavity is a human oral cavity.
E.3 Method E, E.1, or E.2 wherein the composition is contacted with the oral cavity by brushing.
E.4 Method E or E.1-E.2 wherein the composition is contacted with the dental surface by a dental pen or syringe.

Further provided are any of Composition 1, et seq. for use in any of Methods A-E.

As used herein, “inhibition” refers to reduction of stains that would otherwise form or develop subsequent to the time of the treatment. Such inhibition can range from a small but observable or measurable reduction to complete inhibition of subsequent staining, by comparison with an untreated or placebo-treated dental surface.

### EXAMPLES

### Example 1

A randomized, examiner blind clinical study is conducted to assess the clinical efficacy the chlorhexidine delivery system of the present invention. The study is designed to assess the reduction of periodontal outcomes in non-surgical periodontal adults.

The study looks at three separate treatments:
Treatment 1: Treatment 1. Utilizes an in-office syringe containing 0.12% by wt. chlorhexidine gel. Also utilized is a delivery “pen” which also contains a 0.12% by wt. chlorhexidine gel, and a commercial toothpaste (which does not contain CHX) applied with a soft-bristle toothbrush.
Treatment 2: Experimental Gel and Commercial Mouthwash Regimen. Utilizes an in-office syringe containing 0.12% by wt. chlorhexidine gel. Also utilized is a commercial mouthwash which also contains 0.12% by wt. chlorhexidine gluconate, and a commercial toothpaste (which does not contain CHX) applied with a soft-bristle toothbrush.
Treatment 3: Commercial Mouthwash Only Regimen (Positive Control). Utilizes only a commercial mouthwash which also contains 0.12% by wt. chlorhexidine gluconate, and a commercial toothpaste applied with a soft-bristle toothbrush.
Note: The “commercial toothpaste” described above is the same formulation for all treatments and does not contain CHX. The “commercial mouthwash” described above is the same for all treatments it us utilized in.
The formulas used for Treatment 1, Treatment 2 and Treatment 3 are described as follows:

| 0.12% CHX Gel | | Mouthwash with 0.12% CHX | |
|---|---|---|---|
| Ingredient | % by wt. | Ingredient. | % by wt. |
| Water | q.s. | Water | q.s. |
| Humectants | 23.8% | Humectants | 23.8% |
| Chlorhexidine | 0.6386 (0.12% | Chlorhexidine | 0.6386 (0.12% |
| Digluconate | chlorhexidine | Digluconate | chlorhexidine |
| Solution 20% | digluconate) | Solution 20% | digluconate) |
| Nonionic | 0.24 | Nonionic | 0.24 |
| Surfactant | | Surfactant | |
| Cetylpyridinium | 0.015 | Cetylpyridinium | 0.015 |
| Chloride | | Chloride | |
| Hydroxyethyl | 1.75 | pH Adjustment | 0.0004 |
| Cellulose | | Agent | |
| pH Adjustment | 0.0004 | Color and Flavor | 0.1 |
| Agent | | | |
| Color and | 0.1 | | |
| Flavor | | | |
| Total | 100.0 | Total | 100.0 |

Commercial Toothpaste (Does not contain chlorhexidine)

| Ingredient. | % by wt. |
|---|---|
| Water | q.s. |
| Humectants | 16.1 |
| Alkali Pyrophosphate Salt | 0.5 |
| Anionic Surfactant | 5.0 |
| Benzyl Alcohol | 0.3 |
| Carboxymethyl Cellulose | 0.9 |
| pH Adjustment Agent | 0.6 |
| Sodium Monofluorophosphate | 1.1 |
| Precipitated Calcium Carbonate | 41.0 |
| Color and Flavor | 1.2 |
| Total | 100.0 |

The study is designed as a phase II, randomized, examiner-blind, three-cell, parallel-group design. Dental plaque and gingivitis are assessed via Silness and Loe Plaque Index and Loe-Silness Gingival Index for each of the regimen groups. Periodontal parameters include probing pocket depths and clinical attachment level measurements. All subjects are provided with their assigned regimen at baseline. They are instructed to brush their teeth for one minute twice daily (morning and evening) with the toothpaste and toothbrush provided. Subjects in the rinsing regimens were instructed to rinse for 30 seconds with 15 ml of their assigned mouthwash twice daily (morning and evening) after brushing their teeth. Subjects assigned to the gel pen regimen are instructed to brush their teeth followed by use of the gel pen as per instructions provided by the study personnel.

Participants are instructed to repeat the same procedure twice daily for the duration of the study. The use of the mouthwash and gel treatment starts with the periodontal therapy and continues for a period of 2 weeks. After 2 weeks of product use, participants are instructed to discontinue use of their mouthwash/gel and to continue using the toothpaste and toothbrush provided for the duration of the study. Subsequent evaluations are performed at the 2-month examination. After the 2-month evaluation, all subjects discontinued product use and returned to their normal oral hygiene regime.

Generally, the results of the study indicate that the primary endpoint demonstrate significant reductions in dental plaque and gingivitis for the novel chlorhexidine delivery system (“Treatment 1”) as compared to a test regimen comprising of an in-office syringe containing 0.12% chlorhexidine gel, a mouthwash containing 0.12% chlorhexidine gluconate, commercial toothpaste and soft bristle toothbrush (“Treatment 2”) and a Commercial mouthwash regimen including only a commercial mouthwash containing 0.12% chlorhexidine gluconate and commercial toothpaste and a soft bristle toothbrush (“Treatment 3”). The results of the study are detailed in Tables 1-7 described below:

(*Note: A positive value in “% change” or “% difference” indicates a reduction in index scores in that column for Tables 1-7)

TABLE 1
Baseline-Adjusted Subject Mean (SE) Gingival and Plaque
Index Scores at the 2-Week Examination For Subjects
Who Completed the 2-Month Pilot Clinical Study

| | | | | |
|---|---|---|---|---|
| | | % | % | % |
| | Adj. 2-Week | change vs. | Difference vs. | Difference vs |
| Treatment | Mean (S.E.) | baseline | Treatment 2 | Treatment 3 |

Gingival Index

| | | | | |
|---|---|---|---|---|
| Treatment 1 | 0.54 (0.07) | 73.6 | 5.3 | 23.9 |
| Treatment 2 | 0.57 (0.07) | 71.9 | — | 19.7 |
| Treatment 3 | 0.71 (0.07) | 65.4 | — | — |

Plaque Index

| | | | | |
|---|---|---|---|---|
| Treatment 1 | 0.46 (0.07) | 70.2 | 9.8 | 20.7 |
| Treatment 2 | 0.51 (0.07) | 67.7 | — | 12.1 |
| Treatment 3 | 0.58 (0.07) | 63.5 | — | — |
| (Control) | | | | |

Gingival Index

Table 1 presents a summary of the baseline-adjusted mean gingival index scores measured at the 2-week examination.

Comparisons versus baseline: The baseline-adjusted mean gingival index scores at the 2-week examination were 0.54 for subjects assigned to the Treatment 1 Regimen, 0.57 for subjects assigned to the Treatment 2 Regimen and 0.71 for subjects assigned to the Treatment 3 (control) Regimen. The percent changes from baseline were 73.6% for the Treatment 1, 71.9% for the Treatment 2 Regimen and 65.4% for the Treatment 3 Regimen (control), of which all of the regimen groups were statistically significant (p<0.001).

Comparison between treatment groups: Relative to subjects in the Treatment 3 regimen (control), subjects in the Treatment 1 and Treatment 2 groups did show trends in reductions in the Index Score but did not exhibit statistically significant reductions 23.9% (p=0.209) and 19.7% (p=0.342), respectively, in gingival index scores at the 2-week examination. Relative to subjects in the Treatment 2 Regimen, subjects in the Treatment 1 did also show trends in the reduction in the Index Score but did not exhibit a statistically significant reduction 5.3% (p=0.949) in gingival index scores at the 2-week examination.

Plaque Index

Table 1 presents a summary of the baseline-adjusted mean plaque index scores measured at the 2-week examination.

Comparisons versus baseline: The baseline-adjusted mean plaque index scores at the 2-week examination were 0.46 for subjects assigned to the Treatment 1, 0.51 for subjects assigned to the Treatment 2 regimen and 0.58 for subjects assigned to the Treatment 3 regimen. The percent changes from baseline were 70.2% for the Treatment 1 group, 67.7% for the Mouthwash Regimen and 63.5% for the Commercial mouthwash regimen, of which all of the regimen groups were statistically significant (p<0.001).

Comparison between treatment groups: Relative to subjects in the Treatment 3 regimen, subjects in the Treatment 1 and Treatment 2 regimens did show a trend in reduction of the Index Score but did not exhibit statistically significant reductions 20.7% (p=0.475) and 12.1% (p=0.805), respectively, in plaque index scores at the 2-week examination.

Relative to subjects in the Treatment 2 regimen, subjects in the Treatment 1 did show trends in the reduction of the Index Score but did not exhibit a statistically significant reduction 9.8% (p=0.852) in plaque index scores at the 2-week examination.

TABLE 2
Baseline-Adjusted Subject Mean (SE) Gingival Severity and
Plaque Severity Index Scores at the 2-Week Examination For
Subjects Who Completed the 2-Month Pilot Clinical Study

| | | | | |
|---|---|---|---|---|
| | | % | % | % |
| | Adj. 2-Week | change vs. | Difference vs. | Difference vs. |
| Treatment | Mean (S.E.) | baseline | Treatment 2 | Treatment 3 |

Gingival Severity Index

| | | | | |
|---|---|---|---|---|
| Treatment 1 | 0.02 (0.02) | 97.8 | 66.7 | 81.8 |
| Treatment 2 | 0.06 (0.02) | 93.5 | | 45.5 |
| Treatment 3 | 0.11 (0.02) | 88.0 | | |
| (Control) | | | | |

Plaque Severity Index

| | | | | |
|---|---|---|---|---|
| Treatment 1 | 0.06 (0.03) | 89.1 | 33.3 | 50.0 |
| Treatment 2 | 0.09 (0.03) | 85.0 | | 25.9 |
| Treatment 3 | 0.12 (0.03) | 79.3 | | |
| (Control) | | | | |

Gingival Severity Index

Table 2 presents a summary of the baseline-adjusted mean gingival severity index scores measured at the 2-week examination.

Comparisons versus baseline: The baseline-adjusted mean gingival severity index scores at the 2-week examination were 0.02 for subjects assigned to the Treatment 1, 0.06 for subjects assigned to the Treatment 2 Regimen and 0.11 for subjects assigned to the Treatment 3 Regiment. The percent changes from baseline were 97.8% for the Treatment 1 Regimen, 93.5% for the Treatment 2 Regimen and 88.0% for the Treatment 3 regimen, of which all of the regimen groups were statistically significant (p<0.001).

Comparison between treatment groups: Relative to subjects in the Treatment 3 Control Regimen, subjects in the Treatment 1 exhibit a statistically significant reduction of 81.8% (p=0.042) in gingival severity index scores at the 2-week examination. Whereas, relative to subjects in the Treatment 3 control regimen, subjects in the Treatment 2 regimen did show a trend to a reduction in the Index Score but did not exhibit a statistically significant reduction 45.5% (p=0.341) in gingival severity index scores at the 2-week examination.

Relative to subjects in the Treatment 2 regimen, subjects in the Treatment 1 did show a trend in the reduction of the Index Score but did not exhibit a statistically significant reduction 66.7% (p=0.524) in gingival severity index scores at the 2-week examination.

Plaque Severity Index

Table 2 presents a summary of the baseline-adjusted mean plaque severity index scores measured at the 2-week examination.

Comparisons versus baseline: The baseline-adjusted mean plaque severity index scores at the 2-week examination were 0.06 for subjects assigned to the Treatment 1, 0.09 for subjects assigned to the Treatment 2 and 0.12 for subjects assigned to the Treatment 3 (control) Regiment. The percent changes from baseline were 89.1% for the Treatment 1 Regimen, 85.0% for the Treatment 2 regimen and 79.3% for the Treatment 3 regimen (control), of which all of the regimen groups were statistically significant (p<0.001).

Comparison between treatment groups: Relative to subjects in the Treatment 3 (control), subjects in the Treatment 1 and Treatment 2 Regimens did show trends in the reductions of the Index Scores but did not exhibit statistically significant reductions 50.0% (p=0.347) and 25.0% (p=0.734), respectively, in plaque severity index scores at the 2-week examination.

Relative to subjects in the Treatment 2 Regimen, subjects in the Treatment 1 did show a trend in the reduction of the Index Score but did not exhibit a statistically significant reduction 33.3% (p=0.792) in plaque severity index scores at the 2-week examination.

TABLE 3
Baseline-Adjusted Subject Mean (SE) Gingival Interproximal and
Plaque interproximal Index Scores at the 2-Week Examination
For Subjects Who Completed the 2-Month Pilot Clinical Study

| | | | | |
|---|---|---|---|---|
| | | % | % | % |
| | Adj. 2-Week | change vs. | Difference vs. | Difference vs. |
| Treatment | Mean (S.E.) | baseline | Treatment 2 | Treatment 3 |

Gingival Interproximal Index

| | | | | |
|---|---|---|---|---|
| Treatment 1 | 0.68 (0.08) | 67.5 | 5.6 | 21.8 |
| Treatment 2 | 0.72 (0.08) | 65.6 | | 17.2 |
| Treatment 3 | 0.87 (0.08) | 58.8 | | |
| (Control) | | | | |

Plaque Interproximal Index

| | | | | |
|---|---|---|---|---|
| Treatment 1 | 0.63 (0.08) | 62.2 | 5.9 | 14.9 |
| Treatment 2 | 0.67 (0.08) | 60.7 | | 9.5 |
| Treatment 3 | 0.74 (0.08) | 56.0 | | |
| (Control) | | | | |

Gingival Interproximal Index

Table 3 presents a summary of the baseline-adjusted mean gingival interproximal index scores measured at the 2-week examination.

Comparisons versus baseline: The baseline-adjusted mean gingival interproximal index scores at the 2-week examination were 0.68 for subjects assigned to the Treatment 1, 0.72 for subjects assigned to the Treatment 2 Regimen and 0.87 for subjects assigned to the Treatment 3 regimen. The percent changes from baseline were 67.5% for the Treatment 1, 65.6% for the Treatment 2 regiment and 58.8% for the Treatment 3 regimen (control), of which all of the regimen groups were statistically significant (p<0.001).

Comparison between treatment groups: Relative to subjects in the Treatment 3 regimen (control), subjects in the Treatment 1 and Treatment 2 regimens did show trends in the reduction of the Index Scores but did not exhibit statistically significant reductions 21.8% (p=0.223) and 17.2% (p=0.387), respectively, in gingival interproximal index scores at the 2-week examination.

Relative to subjects in the Treatment 2 regimen, subjects in the Treatment 1 did show a trend to a reduction in the Index Score but did not exhibit a statistically significant reduction 5.6% (p=0.933) in gingival interproximal index scores at the 2-week examination.

Plaque Interproximal Index

Table 3 presents a summary of the baseline-adjusted mean plaque interproximal index scores measured at the 2-week examination.

Comparisons versus baseline: The baseline-adjusted mean plaque interproximal index scores at the 2-week examination were 0.63 for subjects assigned to the Treatment 1, 0.67 for subjects assigned to the Treatment 2 Regimen and 0.74 for subjects assigned to the Treatment 3 (control) regimen. The percent changes from baseline were 62.2% for the Treatment 1, 60.7% for the Treatment 2 Regimen and 56.0% for the Treatment 3 regimen, of which all of the regimen groups were statistically significant (p<0.001).

Comparison between treatment groups: Relative to subjects in the Treatment 3 (control) Regimen, subjects in the Treatment 1 and Treatment 2 Regimens did show trends to a reduction in Index Scores but did not exhibit statistically significant reductions 14.9% (p=0.581) and 9.5% (p=0.802), respectively, in plaque interproximal index scores at the 2-week examination.

Relative to subjects in the Treatment 2 Regimen, subjects in the Treatment 1 did show a trend to the a reduction in the Index Score but did not exhibit a statistically significant reduction 5.9% (p=0.931) in plaque interproximal index scores at the 2-week examination.

TABLE 4
Baseline-Adjusted Subject Mean (SE) Gingival and Plaque
Index Scores at the 2-Month Examination For Subjects
Who Completed the 2-Month Pilot Clinical Study

| | | | | |
|---|---|---|---|---|
| | | % | % | % |
| | Adj. 2-Week | change vs. | Difference vs. | Difference vs. |
| Treatment | Mean (S.E.) | baseline | Treatment 2 | Treatment 3 |

Gingival Index

| | | | | |
|---|---|---|---|---|
| Treatment 1 | 0.18 (0.07) | 92 | 43.8 | 61.7 |
| Treatment 2 | 0.32 (0.07) | 84.2 | — | 31.9 |
| Treatment 3 | 0.47 (0.07) | 76.1 | — | — |
| (Control) | | | | |

Plaque Index

| | | | | |
|---|---|---|---|---|
| Treatment 1 | 0.32 (0.07) | 79.5 | 31.9 | 50.8 |
| Treatment 2 | 0.47 (0.07) | 69.6 | — | 27.7 |
| Treatment 3 | 0.65 (0.07) | 58.3 | — | — |
| (Control) | | | | |

Gingival Index

Table 4 presents a summary of the baseline-adjusted mean gingival index scores measured at the 2-month examination.

Comparisons versus baseline: The baseline-adjusted mean gingival index scores at the 2-month examination were 0.18 for subjects assigned to the Treatment 1, 0.32 for subjects assigned to the Treatment 2 Regimen and 0.47 for subjects assigned to the Treatment 3 (control) Regimen. The percent changes from baseline were 92.0% for the Treatment 1 Regimen, 84.2% for the Treatment 2 Regimen and 76.1% for the Treatment 3 mouthwash regimen (control), of which all of the regimen groups were statistically significant (p<0.001).

Comparison between treatment groups: Relative to subjects in the Treatment 3 (control) Regimen, subjects in the Treatment 1 exhibited a statistically significant reduction 61.7% (p=0.009) in gingival index scores at the 2-month examination. Whereas, relative to the subjects in the Treatment 3 regimen, subjects in the Treatment 2 Regimen did show a trend to a reduction in the Index Score but did not exhibit a statistically significant reduction 31.9% (p=0.240) in gingival index scores at the 2-month examination.

Relative to subjects in the Treatment 2 Regimen, subjects in the Treatment 1 did show a trend to a reduction in the Index Score but did not exhibit a statistically significant reduction 43.8% (p=0.306) in gingival index scores at the 2-month examination.

Plaque Index Table 4 presents a summary of the baseline-adjusted mean plaque index scores measured at the 2-month examination.

Comparisons versus baseline: The baseline-adjusted mean plaque index scores at the 2-month examination were 0.32 for subjects assigned to the Treatment 1, 0.47 for subjects assigned to the Treatment 2 Regimen and 0.65 for subjects assigned to the Treatment 3 Regimen (control). The percent changes from baseline were 79.5% for the Treatment 1 Regimen, 69.5% for the Treatment 2 Regimen and 58.3% for the Treatment 3 regimen, of which all of the regimen groups were statistically significant (p<0.001).

Comparison between treatment groups: Relative to subjects in the Treatment 3 (control) Regimen, subjects in the Treatment 1 exhibited a statistically significant reduction 50.8% (p=0.003) in plaque index scores at the 2-month examination. Whereas, relative to the subjects in the Treatment 3 regimen, subjects in the Treatment 2 Regimen demonstrated a trend in the reduction of the Index Score but did not exhibit a statistically significant reduction 27.7% (p=0.141) in plaque index scores at the 2-month examination.

Relative to subjects in the Treatment 2 Regimen, subjects in the Treatment 1 demonstrated a trend in the reduction of the Index Score but did not exhibit a statistically significant reduction 31.9% (p=0.267) in plaque index scores at the 2-month examination.

TABLE 5
Baseline-Adjusted Subject Mean (SE) Gingival Severity and
Plaque Severity Index Scores at the 2-Month Examination For
Subjects Who Completed the 2-Month Pilot Clinical Study

| | | | | |
|---|---|---|---|---|
| | | % | % | % |
| | Adj. 2-Week | change vs. | Difference vs. | Difference vs. |
| Treatment | Mean (S.E.) | baseline | Treatment 2 | Treatment 3 |

Gingival Severity Index

| | | | | |
|---|---|---|---|---|
| Treatment 1 | 0.00 (0.02) | 100 | 100 | 100 |
| Treatment 2 | 0.01 (0.02) | 98.5 | — | 75 |
| Treatment 3 | 0.04 (0.02) | 95.7 | — | — |
| (Control) | | | | |

Plaque Severity Index

| | | | | |
|---|---|---|---|---|
| Treatment 1 | 0.04 (0.02) | 92.7 | 50 | 69.2 |
| Treatment 2 | 0.08 (0.02) | 86.7 | — | 38.5 |
| Treatment 3 | 0.13 (0.02) | 77.6 | — | — |
| (Control) | | | | |

Gingival Severity Index

Table 5 presents a summary of the baseline-adjusted mean gingival severity index scores measured at the 2-month examination.

Comparisons versus baseline: The baseline-adjusted mean gingival severity index scores at the 2-month examination were 0.00 for subjects assigned to the Treatment 1, 0.01 for subjects assigned to the Treatment 2 Regimen and 0.04 for subjects assigned to the Treatment 3 Regimen (control). The percent changes from baseline were 100.0% for the Treatment 1 Regimen, 98.5% for the Treatment 2 Regimen and 95.7% for the Treatment 3 regimen (control), of which all of the regimen groups were statistically significant (p<0.001).

Comparison between treatment groups: Relative to subjects in the Treatment 3 (control) Regimen, subjects in the Treatment 1 and Treatment 2 demonstrated trends in the reduction of the Index Scores but did not exhibit statistically significant reductions 100.0% (p=0.279) and 75.0% (p=0.543), respectively, in gingival severity index scores at the 2-month examination.

Relative to subjects in the Treatment 2 Regimen, subjects in the Treatment 1 demonstrated a trend in the reduction of the Index Score but did not exhibit a statistically significant reduction 100.0% (p=0.876) in gingival severity index scores at the 2-month examination.

Plaque Severity Index

Table 5 presents a summary of the baseline-adjusted mean plaque severity index scores measured at the 2-month examination.

Comparisons versus baseline: The baseline-adjusted mean plaque severity index scores at the 2-month examination were 0.04 for subjects assigned to the Treatment 1, 0.08 for subjects assigned to the Treatment 2 and 0.13 for subjects assigned to the Treatment 3 (control) Regimen. The percent changes from baseline were 92.7% for the Treatment 1 Regimen, 86.7% for the Treatment 2 Regimen and 77.6% for the Treatment 3 regimen, of which all of the regimen groups were statistically significant (p<0.001).

Comparison between treatment groups: Relative to subjects in the Treatment 3 (control) Regimen, subjects in the Treatment 1 exhibited a statistically significant reduction 69.2% (p=0.012) in plaque severity index scores at the 2-month examination. Whereas, relative to the subjects in the Treatment 3 regimen (control), subjects in the Treatment 2 Regimen demonstrated a trend in the reduction of the Index Score but did not exhibit a statistically significant reduction 38.5% (p=0.235) in plaque severity index scores at the 2-month examination.

Relative to subjects in the Treatment 2 Regimen, subjects in the Treatment 1 demonstrated a trend in the reduction of the Index Score but did not exhibit a statistically significant reduction 50.0% (p=0.378) in plaque severity index scores at the 2-month examination.

TABLE 6
Baseline-Adjusted Subject Mean (SE) Gingival Interproximal and
Plaque Interproximal Index Scores at the 2-Week Examination
For Subjects Who Completed the 2-Month Pilot Clinical Study

| | | | | |
|---|---|---|---|---|
| | | % | % | % |
| | Adj. 2-Week | change vs. | Difference vs. | Difference vs. |
| Treatment | Mean (S.E.) | baseline | Treatment 2 | Treatment 3 |

Gingival Interproximal Index

| | | | | |
|---|---|---|---|---|
| Treatment 1 | 0.22 (0.08) | 89.5 | 46.3 | 62.7 |
| Treatment 2 | 0.41 (0.08) | 80.9 | — | 30.5 |
| Treatment 3 | 0.59 (0.08) | 71.6 | — | — |
| (Control) | | | | |

Plaque Interproximal Index

| | | | | |
|---|---|---|---|---|
| Treatment 1 | 0.45 (0.08) | 73.8 | 25.0 | 44.4 |
| Treatment 2 | 0.60 (0.08) | 64.2 | — | 25.9 |
| Treatment 3 | 0.81 (0.08) | 51.2 | — | — |
| (Control) | | | | |

Gingival Interproximal Index

Table 6 presents a summary of the baseline-adjusted mean gingival interproximal index scores measured at the 2-month examination.

Comparisons versus baseline: The baseline-adjusted mean gingival interproximal index scores at the 2-month examination were 0.22 for subjects assigned to the Treatment 1, 0.41 for subjects assigned to the Treatment 2 Regimen and 0.59 for subjects assigned to the Treatment 3 regimen (control). The percent changes from baseline were 89.5% for the Treatment 1, 80.9% for the Treatment 2 Regimen and 71.6% for the Treatment 3 Regimen (control), of which all of the regimen groups were statistically significant (p<0.001).

Comparison between treatment groups: Relative to subjects in the Treatment 3 (control) Regimen, subjects in the Treatment 1 exhibited a statistically significant reduction 62.7% (p=0.008) in gingival interproximal index scores at the 2-month examination. Whereas, relative to the subjects in the Commercial mouthwash regimen, subjects in the Treatment 2 Regimen demonstrated a trend in the reduction of the Index Score but did not exhibit a statistically significant reduction 30.5% (p=0.269) in gingival interproximal index scores at the 2-month examination.

Relative to subjects in the Treatment 2 Regimen, subjects in the Treatment 1 exhibited a trend in the reduction of the Index Score but did not exhibit a statistically significant reduction 46.3% (p=0.258) in gingival interproximal index scores at the 2-month examination.

Plaque Interproximal Index

Table 6 presents a summary of the baseline-adjusted mean plaque interproximal index scores measured at the 2-month examination.

Comparisons versus baseline: The baseline-adjusted mean plaque interproximal index scores at the 2-month examination were 0.45 for subjects assigned to the Treatment 1 Regimen, 0.60 for subjects assigned to the Treatment 2 Regimen and 0.81 for subjects assigned to the Treatment 3 regimen (control). The percent changes from baseline were 73.8% for the Treatment 1, 64.2% for the Treatment 2 Regimen and 51.2% for the Treatment 3 regimen (control), of which all of the regimen groups were statistically significant (p<0.001).

Comparison between treatment groups: Relative to subjects in the Treatment 3 Regimen (control), subjects in the Treatment 1 exhibited a statistically significant reduction 44.4% (p=0.006) in plaque interproximal index scores at the 2-month examination. Whereas, relative to the subjects in the Commercial mouthwash regimen, subjects in the Treatment 2 Regimen demonstrated a trend in the reduction of the Index Score but did not exhibit a statistically significant reduction 29.5% (p=0.151) in plaque interproximal index scores at the 2-month examination.

Relative to subjects in the Treatment 2 Regimen, subjects in the Treatment 1 demonstrated a trend in the reduction of the Index Score but did not exhibit a statistically significant reduction 25.0% (p=0.379) in plaque interproximal index scores at the 2-month examination.

TABLE 7
Mean (+/−SD) reduction of probing depth and gain
in clinical attachment level at 2 weeks and 2 months
of periodontal pockets with PD ≥5 mm in all groups.

| | | | |
|---|---|---|---|
| | | Δ at 2 weeks | Δ at 2 weeks |
| Treatment | N | (vs. baseline) | (vs. baseline) |

Probing Depth

| | | | |
|---|---|---|---|
| Treatment 1 | 15 | 2.6 +/− 0.7 | 3.2 +/− 0.5 |
| Experimental gel and | 15 | 2.7 +/− 0.8 | 3.2 +/− 0.5 |
| commercial mouthwash | | | |
| regimen | | | |
| Commercial mouthwash | 15 | 2.4 +/− 0.7 | 3.1 +/− 0.5 |
| regimen (Control) | | | |
| p-value | | 0.544 | 0.826 |

Clinical Attachment Level

| | | | |
|---|---|---|---|
| Treatment 1 | 15 | 2.7 +/− 1.0 | 3.3 +/− 0.7 |
| Experimental gel and | 15 | 2.7 +/− 0.8 | 3.3 +/− 0.5 |
| commercial mouthwash | | | |
| regimen | | | |
| Commercial mouthwash | 15 | 2.4 +/− 0.7 | 3.1 +/− 0.5 |
| regimen (Control) | | | |
| p-value | | 0.589 | 0.753 |

Probing Depth

Comparison between treatment groups: Table 7 shows probing depth reduction at 2-week and 2-month evaluation after non-surgical periodontal treatment, for each regimen Subjects assigned to the Treatment 1, at the 2-week examination, exhibited a mean probing depth reduction of 2.6 (+/−0.7) mm, while Treatment 2 Regimen and Treatment 3 (control) regimen showed 2.7(+/−0.8) mm and 2.4 (+/−0.7) mm, respectively, with no statistically significant differences among regimens (p=0.544). Similarly, at the 2-month examination, no statistically significant difference among groups were noted (p=0.826) with Treatment 1 subjects exhibiting a mean probing depth reduction of 3.2(+/−0.5) mm and Treatment 2 Regimen and Treatment 3 regimen (control) showing 3.2(+/−0.5) mm and 3.1(+/−0.5) mm, respectively.

Clinical Attachment Levels

Comparison between treatment groups: Table 7 shows the clinical attachment level gains after non-surgical periodontal treatment, at 2-week and 2-month evaluation for each regimen, At the 2-week examination, Treatment 1 exhibited a mean clinical attachment level gain of 2.7(+/−1.0) mm, while Treatment 2 Regimen and Treatment 3 (control) regimen showed 2.7(+/−0.8 mm and 2.4(+/−0.7) mm, respectively, with no statistically significant difference among the regimens (p=0.589). At the 2-month examination, no statistically significant differences among groups were noted (p=0.753). Treatment 1 showed a mean clinical attachment level gain of 3.3(+/−0.7) mm while Treatment 2 and the Treatment 3 (control) regimen showed 3.3(+/−0.5) mm and 3.1(+/−0.5) mm, respectively.

### Example 2

The following are representative formulations of the present invention:

Formula A

| Ingredient | % by wt. |
|---|---|
| Water | q.s. |
| Humectants | 23.8% |
| Chlorhexidine | 0.6386 (0.12% |
| Digluconate Solution 20% | chlorhexidine digluconate) |
| Nonionic Surfactant | 0.24 |
| Cetylpyridinium Chloride | 0.015 |
| Hydroxyethyl Cellulose | 1.75 |
| pH Adjustment Agent | 0.0004 |
| Color and Flavor | 0.5 |
| Total | 100.0 |

Formula B

| Ingredient | % by wt. |
|---|---|
| Water | q.s. |
| Humectants | 24% |
| Chlorhexidine | 1.065 (0.2% |
| Digluconate Solution 20% | chlorhexidine digluconate) |
| Nonionic Surfactant | 0.24 |
| Cetylpyridinium Chloride | 0.015 |
| Hydroxyethyl Cellulose | 1.75 |
| pH Adjustment Agent | 0.0004 |
| Color and Flavor | 0.5 |
| Total | 100.0 |

1. An oral care composition comprising:
(i) an effective amount of a bisbiguanide in free or orally acceptable salt form;
(ii) an effective amount of a nonionic cellulose ether; and
(iii) water.

2. (canceled)

3. The oral care composition of claim 1, wherein the bisbiguanide is chlorhexidine in free or orally acceptable salt form.

4. (canceled)

5. The oral care composition of claim 1, wherein the bisbiguanide is chlorhexidine digluconate.

6. The oral care composition of claim 1, wherein the composition comprises an orally acceptable cationic active agent selected from: quaternary ammonium surfactants, amino acids, metal cations, guanidinium polymers, and combinations thereof.

7. The oral care composition of claim 6, wherein the orally acceptable cationic active agent comprises an agent selected from one or more of: cetyl pyridinium chloride (CPC)); arginine; antimicrobial guanidinium polymers; a source of zinc, and combinations thereof.

8. (canceled)

9. The oral care composition of claim 7, wherein the orally acceptable cationic active agent comprises cetyl pyridinium chloride (CPC).

10. The oral care composition of claim 1, wherein the bisbiguanide comprises chlorhexidine gluconate in an amount of 0.1 to 0.3% by wt. of the total composition.

11. The oral care composition of claim 10, wherein the chlorhexidine digluconate is present in an amount of about 0.12% by wt. of the total composition.

12. The oral care composition of claim 10, wherein the chlorhexidine digluconate is present in an amount of about 0.20% by wt. of the total composition.

13. The oral care composition of claim 1, wherein the nonionic cellulose ether is selected from the group consisting of: ethyl cellulose, hydroxyethyl cellulose, methyl cellulose, sodium carboxy methyl cellulose, and benzyl cellulose.

14. The oral care composition of claim 13, wherein the nonionic cellulose ether comprises hydroxyethyl cellulose (HEC).

15. The oral care composition of claim 14, wherein the hydroxyethyl cellulose is in an amount of from 0.5 wt. % to 3 wt. % of the total composition.

16. The oral care composition of claim 15, wherein the hydroxyethyl cellulose is in an amount of from about 1.75 wt. % of the total composition.

17. (canceled)

18. (canceled)

19. The composition of claim 1, wherein the composition is selected from: a mouthwash, toothpaste, tooth gel, tooth powder, non-abrasive gel, mousse, foam, mouth spray, lozenge, oral tablet, and dental implement.

20. The composition of claim 19, wherein the composition is a tooth gel.

21. The oral care composition of claim 1, wherein the oral care composition is a gel and comprises:
An effective amount of chlorhexidine in free or orally acceptable salt form;
Cetylpyridinium chloride;
An effective amount of hydroxyethyl cellulose; and
wherein the water content is from 60%-90% by wt. of the composition.

22. The oral care composition of claim 1, wherein the oral care composition is a gel and comprises:
An effective amount of chlorhexidine in free or orally acceptable salt form;
Cetylpyridinium chloride;
An effective amount of hydroxyethyl cellulose;
Glycerin and Sorbitol; and
wherein the water content is from 60%-90% by wt. of the composition.

23. A method of treatment and/or inhibition of a gingivitis, chemical stain, plaque, and/or tartar on a dental surface, comprising contacting the dental surface with the oral care composition of claim 1.

24. (canceled)

25. (canceled)

26. (canceled)

27. A delivery system for administration of the oral care composition of claim 1 to a patient in need thereof, wherein the delivery system comprises a syringe and a dental pen.

28. The delivery system of claim 27, wherein the patient in need thereof receives the administration of the composition post-surgery.
